(19) United States
(12) Patent Application Publication
(10) Pub. No.: US 2015/0291574 A1
(43) Pub. Date: Oct. 15, 2015

(54) NOVEL POLYMORPHS OF AZILSARTAN

(71) Applicants: Bandi PARTHASARADHI REDDY, (US); Kura RATHNAKAR REDDY, Hyderabad, Andhra Pradesh (IN); Dasari MURALIDHARA REDDY, (US); Matta RAMAKRISHNA REDDY, (US); HETERO RESEARCH FOUNDATION, Andhrapradesh, Hyderabad (IN)

(72) Inventors: Bandi Parthasaradhi Reddy, Hyderabad (IN); Kura Rathnakar Reddy, Hyderabad (IN); Dasari Muralidhara Reddy, Hyderabad (IN); Matta Ramakrishna Reddy, Hyderabad (IN); Bandi Vamsi Krishna, Hyderabad (IN)

(21) Appl. No.: 14/589,575

(22) PCT Filed: Jul. 8, 2013
(86) PCT No.: PCT/IN13/00416

§ 371 Date: Jan. 5, 2015

(30) Foreign Application Priority Data
Jul. 9, 2012 (IN) 2760/CHE/2012

(51) Int. Cl. C07D 413/14

(52) U.S. Cl. CPC C07D 413/14; C07B 2200/13

(57) ABSTRACT

The present invention provides a novel crystalline Form of azilsartan acid, process for its preparation and pharmaceutical compositions comprising it. The present invention also provides a novel crystalline Form of azilsartan medoxomil potassium, process for its preparation and pharmaceutical compositions comprising it.

This application is a national stage application of PCT/IN2013/000416 which claims the benefit of Indian patent Application No. 2760/CHE/2012, filed on Jul. 9, 2012, which is incorporated herein by reference.

### FIELD OF THE INVENTION

The present invention provides a novel crystalline Form of azilsartan acid, process for its preparation and pharmaceutical compositions comprising it. The present invention also provides a novel crystalline Form of azilsartan medoxomil potassium, process for its preparation and pharmaceutical compositions comprising it.

### BACKGROUND OF THE INVENTION

Azilsartan medoxomil is chemically, (5-methyl-2-oxo-1,3-dioxol-4-yl)methyl-2-ethoxy-1-([2′-(5-oxo-4,5-dihydro-1,2,4-oxadiazol-3-yl)biphenyl-4-yl]methyl)-1H-benzimidazole-7-carboxylate and has the structural formula:

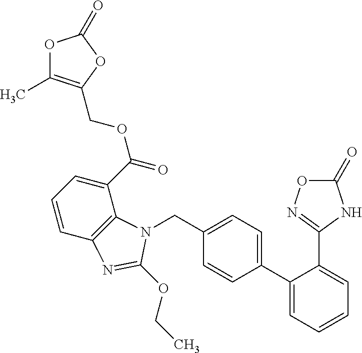 [SMILES: CCOc1nc2cccc(C(=O)OCc3oc(=O)oc3C)c2n1Cc1ccc(-c2ccccc2-c2noc(=O)[nH]2)cc1]

Azilsartan (INN, codenamed TAK-536) is an angiotensin II receptor antagonist used in the treatment of hypertension. It is marketed by Takeda Pharmaceuticals under the brand name EDARBI®.

Azilsartan acid and its process were disclosed in U.S. Pat. No. 5,243,054 ('054 patent).

Azilsartan medoxomil and its potassium salt were disclosed in U.S. Pat. No. 7,157,584 ('584 patent).

Polymorphism is defined as “the ability of a substance to exist as two or more crystalline phases that have different arrangement and/or conformations of the molecules in the crystal Lattice. Thus, in the strict sense, polymorphs are different crystalline structures of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and Infrared spectrometry (IR).

Solvent medium and mode of crystallization play very important role in obtaining one polymorphic Form over the other.

Azilsartan medoxomil and its potassium salt can exist in different polymorphic Forms, which may differ from each other in terms of stability, physical properties, spectral data and methods of preparation.

Process for the preparation of azilsartan acid was disclosed in the '054 patent. According to the patent, crystalline solid of azilsartan acid was obtained by reacting 2-ethoxy-1-[2′-(2,5-dihydro-5-oxo-1,2,4-oxadiazol-3-yl)biphenyl-4-yl]methyl]benzimidazole-7-carboxylate in methanol with lithium hydroxide in water, pH was adjusted to 3.0 with hydrochloric acid and then concentrated to obtain a residue. To the residue was added chloroform and water and then the organic layer was dried, and then concentrated to provide a crystalline product. The crystalline product was recrystallized with ethyl acetate. The crystalline azilsartan acid obtained by the process of the prior art is herein after designated as azilsartan acid crystalline Form I. The powdered x-ray diffractogram (PXRD) of azilsartan acid crystalline Form I is shown in FIG. 1. Crystalline Form I is characterized by peaks in the powder x-ray diffraction spectrum having 2θ angle positions at about 11.3, 14.7, 14.9, 19.9, 21.5, 22.0 and 24.7±0.2 degrees.

Process for the preparation of azilsartan medoxomil potassium was disclosed in the '584 patent. According to the patent, crystalline solid of azilsartan acid was obtained by reacting (5-methyl-2-oxo-1,3-dioxol-4-yl)methyl-2-ethoxy-1-([2′-(5-oxo-4,5-dihydro-1,2,4-oxadiazol-3-yl)biphenyl-4-yl]methyl)-1H-benzimidazole-7-carboxylate with potassium 2-ethylhexanoate in acetone at low temperature for overnight. The crystalline azilsartan medoxomil potassium obtained by the process of the prior art is herein after designated as azilsartan medoxomil potassium crystalline Form I. The powdered x-ray diffractogram (PXRD) of azilsartan medoxomil potassium crystalline Form I is shown in FIG. 3. Crystalline Form I is characterized by peaks in the powder x-ray diffraction spectrum having 2θ angle positions at about 6.0, 6.2, 14.7, 15.0 and 22.8±0.2 degrees.

We have found a novel crystalline Form of azilsartan acid. The novel Form is stable, reproducible and so, suitable for pharmaceutical preparations.

We have also found a novel crystalline Form of azilsartan medoxomil potassium. The novel Form is stable, reproducible and so, suitable for pharmaceutical preparations.

Thus, an object of the present invention is to provide a novel crystalline Form of azilsartan acid, process for its preparation and pharmaceutical compositions comprising it.

Another object of the present invention is to provide a novel crystalline Form of azilsartan medoxomil potassium, process for its preparation and pharmaceutical compositions comprising it.

### SUMMARY OF THE INVENTION

In one aspect, the present invention provides a crystalline Form of azilsartan acid designated as Form II characterized by peaks in the powder x-ray diffraction spectrum having 2θ angle positions at about 9.1, 12.7, 18.6, 19.3, 21.4 and 23.5±0.2 degrees.

In another aspect, the present invention provides a process for the preparation of azilsartan acid crystalline Form II, which comprises:

a) dissolving 2-ethoxy-1-[2′-(2,5-dihydro-5-oxo-1,2,4-oxadiazol-3-yl)biphenyl-4-yl]methyl]benzimidazole-7-carboxylate in methanol;
b) adding a solution of sodium hydroxide or potassium hydroxide in water;
c) heating the contents at reflux;
d) adjusting the pH of the reaction mass to about 2.0 to 3.0 with hydrochloric acid;
e) isolating the solid;
f) slurring the solid obtained in step (e) with a chlorinated solvent and water;
g) isolating the wet solid;
h) slurring the wet solid obtained in step (g) with an ester solvent and water; and
i) isolating azilsartan acid crystalline Form II.

In another aspect, the present invention provides a pharmaceutical composition comprising crystalline Form II of azilsartan acid and pharmaceutically acceptable excipients.

In another aspect, the present invention provides a crystalline Form of azilsartan medoxomil potassium designated as Form II characterized by peaks in the powder x-ray diffraction spectrum having 2θ angle positions at about 6.3, 13.4, 14.4, 14.7 and 22.8±0.2 degrees.

In another aspect, the present invention provides a process for the preparation of azilsartan medoxomil potassium crystalline Form II, which comprises:

a) suspending azilsartan medoxomil in a solvent;

b) heating the suspension obtained in step (a) at above 40° C.;

c) cooling the solution obtained in step (b) at room temperature;

d) adding potassium 2-ethylhexanoate in a solvent to the solution;

e) maintaining the reaction mass at room temperature; and

f) isolating azilsartan medoxomil potassium crystalline Form II.

Yet in another aspect, the present invention provides a pharmaceutical composition comprising crystalline Form II of azilsartan medoxomil potassium and pharmaceutically acceptable excipients.

### BRIEF DESCRIPTION OF THE DRAWINGS

FIG. 1 is an X-ray powder diffraction spectrum of azilsartan acid crystalline Form I.

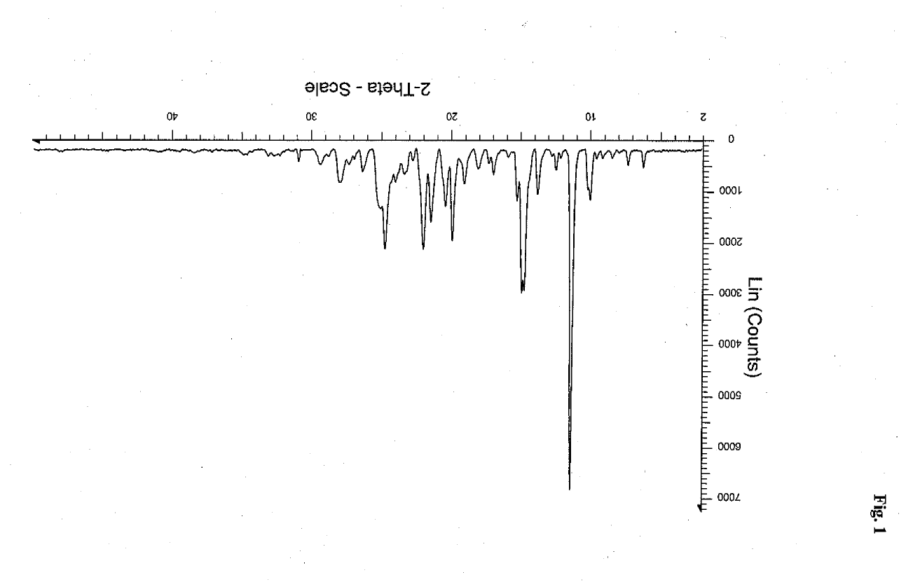

FIG. 2 is an X-ray powder diffraction spectrum of azilsartan acid crystalline Form II.

FIG. 3 is an X-ray powder diffraction spectrum of azilsartan medoxomil potassium crystalline Form I.

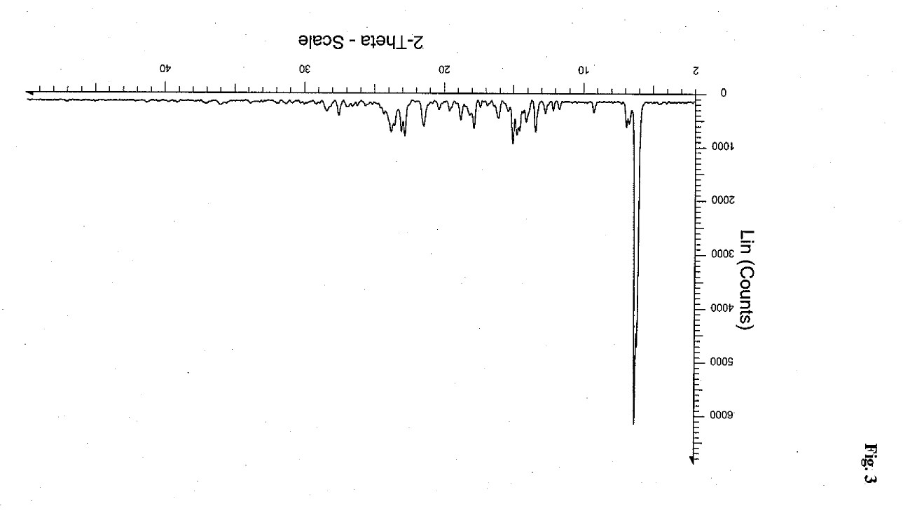

FIG. 4 is an X-ray powder diffraction spectrum of azilsartan medoxomil potassium crystalline Form II.

X-ray powder diffraction spectrum was measured on a bruker axs D8 advance X-ray powder diffractometer having a copper-Kα radiation. Approximately 500 gm of sample was gently flattered on a sample holder and scanned from 2 to 50 degrees two-theta, at 0.020 degrees two theta per step and a step time of 1 second. The sample was simply placed on the sample holder. The sample was rotated at 30 rpm at a voltage 40 KV and current 35 mA.

### DETAILED DESCRIPTION OF THE INVENTION

The term “room temperature” refers to temperature at about 25 to 35° C.

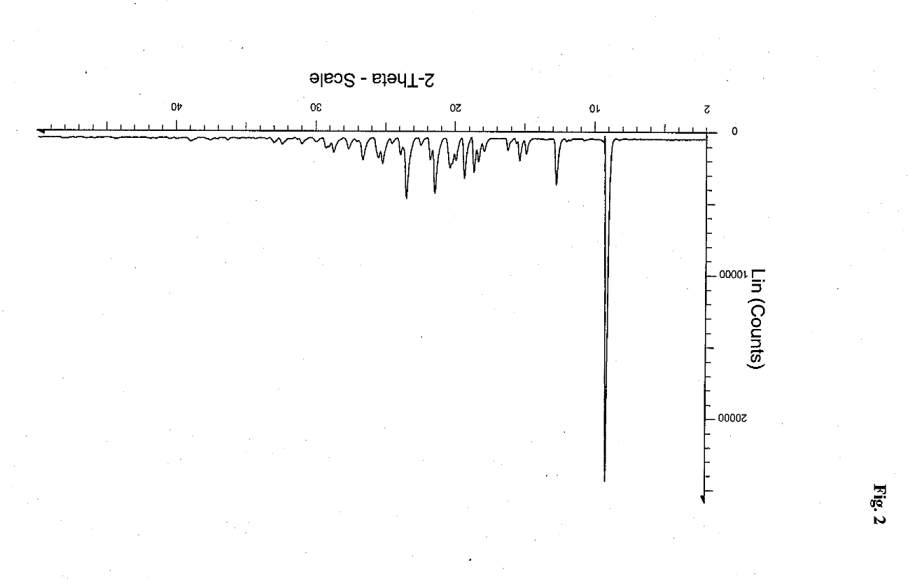

According to one aspect of the present invention, there is provided a crystalline Form of azilsartan acid designated as Form II characterized by peaks in the powder x-ray diffraction spectrum having 2θ angle positions at about 9.1, 12.7, 18.6, 19.3, 21.4 and 23.5±0.2 degrees. The powdered x-ray diffractogram (PXRD) of azilsartan acid crystalline Form II is shown in FIG. 2.

According to another aspect of the present invention, there is provided a process for the preparation of azilsartan acid crystalline Form II, which comprises:
a) dissolving 2-ethoxy-1-[2′-(2,5-dihydro-5-oxo-1,2,4-oxadiazol-3-yl)biphenyl-4-yl]methyl]benzimidazole-7-carboxylate in methanol;
b) adding a solution of sodium hydroxide or potassium hydroxide in water;
c) heating the contents at reflux;
d) adjusting the pH of the reaction mass to about 2.0 to 3.0 with hydrochloric acid;
e) isolating the solid;
f) slurring the solid obtained in step (e) with a chlorinated solvent and water;
g) isolating the wet solid;
h) slurring the wet solid obtained in step (g) with an ester solvent and water; and
i) isolating azilsartan acid crystalline Form II.

The solid may be isolated in step (e) by methods known such as filtration or centrifugation.

The chlorinated solvent used in step (f) may preferably be a solvent or mixture of solvents selected from methylene chloride, chloroform, carbontetrachloride and ethylene dichloride, and more preferably the chlorinated solvent is chloroform.

Isolation of wet solid in step (g) can be performed by conventional methods such as cooling, removal of solvents, concentrating the reaction mass, adding an anti-solvent, extraction with a solvent and the like.

The ester solvent used in step (h) may preferably be a solvent or mixture of solvents selected from ethyl acetate, methyl acetate, isopropyl acetate, tert-butyl methyl acetate and ethyl formate, and more preferably the ester solvent is ethyl acetate.

Isolation of azilsartan acid crystalline Form II in step (i) can be performed by conventional methods such as cooling, removal of solvents, concentrating the reaction mass, adding an anti-solvent, extraction with a solvent and the like.

The azilsartan acid crystalline Form II of the present invention may also serve as intermediate for preparation of azilsartan medoxomil or salt of azilsartan medoxomil.

According to another aspect of the present invention, there is provided a pharmaceutical composition comprising crystalline Form II of azilsartan acid and pharmaceutically acceptable excipients, and optionally other therapeutic ingredients. The crystalline Form II may preferably be formulated into tablets, capsules, suspensions, dispersions, injectables or other pharmaceutical forms.

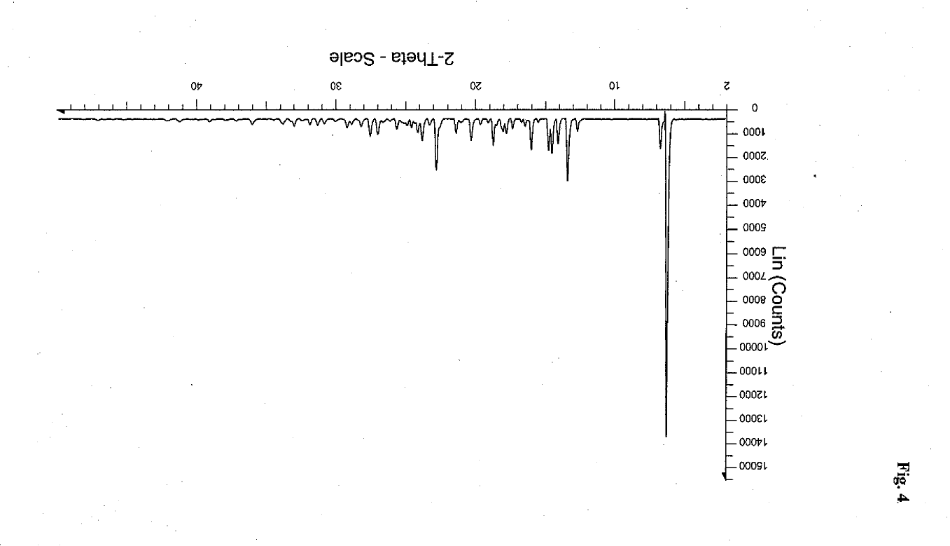

According to another aspect of the present invention, there is provided a crystalline Form of azilsartan medoxomil potassium designated as Form II characterized by peaks in the powder x-ray diffraction spectrum having 2θ angle positions at about 6.3, 13.4, 14.4, 14.7 and 22.8±0.2 degrees. The powdered x-ray diffractogram (PXRD) of azilsartan medoxomil potassium crystalline Form II is shown in FIG. 4.

The azilsartan medoxomil potassium crystalline form II may be identified and differentiated from the known polymorphs by its characteristic PXRD pattern. Thus, for example, a peak at 6.0 degrees 2θ is absent in the PXRD of the azilsartan medoxomil potassium crystalline form II of the present invention, but is present in the PXRD of the crystalline form I of azilsartan medoxomil potassium described in the U.S. Pat. No. 7,157,584.

According to another aspect of the present invention, there is provided a process for the preparation of azilsartan medoxomil potassium crystalline Form II, which comprises:

a) suspending azilsartan medoxomil in a solvent;

b) heating the suspension obtained in step (a) at above 40° C.;

c) cooling the solution obtained in step (b) at room temperature;

d) adding potassium 2-ethylhexanoate in a solvent to the solution;

e) maintaining the reaction mass at room temperature; and

f) isolating azilsartan medoxomil potassium crystalline Form II.

The solvent used in step (a) and step (d) may preferably be a solvent or mixture of solvents selected from acetone, methyl ethyl ketone, methyl isobutyl ketone, diethyl ketone, ethyl acetate, methyl acetate, isopropyl acetate, tert-butyl methyl acetate and ethyl formate. More preferably the solvents are acetone, methyl ethyl ketone and ethyl acetate.

The reaction in step (b) may preferably be heated at about 45 to 65° C.

The azilsartan medoxomil potassium crystalline Form II may be isolated in step (f) by methods known such as filtration or centrifugation.

According to another aspect of the present invention, there is provided a pharmaceutical composition comprising crystalline Form II of azilsartan medoxomil potassium and pharmaceutically acceptable excipients, and optionally other therapeutic ingredients. The crystalline Form II may preferably be formulated into tablets, capsules, suspensions, dispersions, injectables or other pharmaceutical forms.

The contents of azilsartan acid and azilsartan medoxomil potassium are determined by High performance liquid chromatography (HPLC).

The invention will now be further described by the following examples, which are illustrative rather than limiting.

### REFERENCE EXAMPLES

### Reference Example 1

### Preparation of Azilsartan Acid Crystalline Form I

To a mixture of lithium hydroxide (0.5 gm), water (10 ml) and methanol (120 ml) was added 2-ethoxy-1-[2′-(2,5-dihydro-5-oxo-1,2,4-oxadiazol-3-yl)biphenyl-4-yl]methyl]benzimidazole-7-carboxylate (1.7 gm) at room temperature. The reaction mass was heated to reflux and maintained for 3 hours. The reaction mass was then cooled to room temperature and pH was adjusted to 3.0 with hydrochloric acid (1N). The solvent was distilled off under vacuum at below 45° C. to provide a residual solid. To the residual solid was added chloroform (500 ml) and water (200 ml) under stifling. The separated organic layer was dried with sodium sulfate and then concentrated to provide a residual solid. To the residual solid was added ethyl acetate (5 ml) and stirred for 15 minutes at 40° C. The contents were then cooled to room temperature and stirred for 30 minutes. The separated solid was filtered and then dried to provide 0.9 gm of azilsartan acid crystalline Form I.

Chromatographic purity: 98.64%.

### Reference Example 2

### Preparation of Azilsartan Medoxomil Potassium Crystalline Form I

Azilsartan medoxomil (6 gm) was dissolved in acetone (110 ml) and then heated to 50° C. for 15 minutes to provide a clear solution. The solution was then cooled to 0° C. and then added a solution of potassium 2-ethylhexanoate (1.85 gm) in acetone (22 ml) slowly for 30 minutes. The reaction mass was maintained for 14 hours at 0° C. and filtered. The solid obtained was dried to provide 3 gm of azilsartan medoxomil potassium crystalline Form I.

Chromatographic purity: 98.1%.

### EXAMPLES

### Example 1

### Preparation of Azilsartan Acid Crystalline Form II

2-Ethoxy-1-[2′-(2,5-dihydro-5-oxo-1,2,4-oxadiazol-3-yl)biphenyl-4-yl]methyl]benzimidazole-7-carboxylate (166 gm) was dissolved in methanol (1600 ml) and then added a solution of sodium hydroxide (50 gm) in water (166 ml) at room temperature. The reaction mass was then heated to reflux and maintained for 1 hour 30 minutes. The reaction mass was treated with carbon and filtered through hi-flow bed. The pH of the filtrate thus obtained was adjusted to 2.5 with hydrochloric acid (20%) at 15 to 20° C. The reaction mass was stirred for 1 hour at room temperature and then cooled to 0 to 5° C. The contents were stirred for 1 hour at 0 to 5° C., filtered and then dried to provide a solid. To the solid was added chloroform (1080 ml) and water (410 ml) under stifling. The contents were heated to 40 to 45° C. and maintained for 30 minuets. The reaction mass was then cooled to 0 to 5° C., maintained for 30 minutes and filtered to provide a wet solid. To the wet solid was added ethyl acetate (1160 ml) and water (500 ml) and then heated to reflux. The solution was maintained for 30 minutes at reflux and then cooled to 0 to 5° C. The contents were stirred for 30 minutes at 0 to 5° C. and filtered. The solid obtained was dried to provide 127 gm of azilsartan acid crystalline Form II.

Chromatographic purity: 99.35%.

### Example 2

### Preparation of Azilsartan Medoxomil Potassium Crystalline Form II

Azilsartan medoxomil (62 gm) was dissolved in acetone (1560 ml) and then heated to 45 to 50° C. The contents were stirred for 1 hour to provide a clear solution and then treated with activated carbon. The solution was then cooled to 0° C. and then added a solution of potassium 2-ethylhexanoate (18.6 gm) in acetone (112 ml) slowly for 20 minutes. The temperature of the reaction mass was raised to room temperature and stirred for 20 hours. The reaction mass was then cooled to 0 to 5° C., stirred for 1 hour at 0 to 5° C. and filtered. The solid obtained was dried to provide 46 gm of azilsartan medoxomil potassium crystalline Form II.

Chromatographic purity: 99.3%

### Example 3

### Preparation of Azilsartan Medoxomil Potassium Crystalline Form II

Azilsartan medoxomil (10 gm) was dissolved in ethyl acetate (500 ml) and then heated to 50 to 60° C. The contents were stirred for 1 hour at 50 to 60° C. to provide a clear solution and then cooled to room temperature. To the solution was added a solution of potassium 2-ethylhexanoate (3 gm) in ethyl acetate (20 ml) slowly for 20 minutes. The reaction mass was stirred for 18 hours at room temperature and then cooled to 0 to 5° C. The contents were stirred for 1 hour at 0 to 5° C. and filtered. The solid obtained was dried to provide 5 gm of azilsartan medoxomil potassium crystalline Form II.

Chromatographic purity: 99.94%.

We claim:

1. Azilsartan acid crystalline Form II, characterized by peaks in the powder x-ray diffraction spectrum having 2θ angle positions at about 9.1, 12.7, 18.6, 19.3, 21.4 and 23.5±0.2 degrees.

2. Azilsartan acid crystalline Form II, characterized by an x-ray powder diffractogram as shown in FIG. 2.

3. A process for the preparation of azilsartan acid crystalline Form II as claimed in claim 1, which comprises:
a. dissolving 2-ethoxy-1-[2′-(2,5-dihydro-5-oxo-1,2,4-oxadiazol-3-yl)biphenyl-4-yl]methyl]benzimidazole-7-carboxylate in methanol;
b. adding a solution of sodium hydroxide or potassium hydroxide in water;
c. heating the contents at reflux;
d. adjusting the pH of the reaction mass to about 2.0 to 3.0 with hydrochloric acid;
e. isolating the solid;
f. slurring the solid obtained in step (e) with a chlorinated solvent and water;
g. isolating the wet solid;
h. slurring the wet solid obtained in step (g) with an ester solvent and water; and
i. isolating azilsartan acid crystalline Form II.

4. The process as claimed in claim 3, wherein the chlorinated solvent used in step (f) is a solvent or mixture of solvents selected from methylene chloride, chloroform, carbontetrachloride and ethylene dichloride.

5. The process as claimed in claim 3, wherein the ester solvent used in step (h) is a solvent or mixture of solvents selected from ethyl acetate, methyl acetate, isopropyl acetate, tert-butyl methyl acetate and ethyl formate.

6. Azilsartan medoxomil potassium crystalline Form II, characterized by peaks in the powder x-ray diffraction spectrum having 2θ angle positions at about 6.3, 13.4, 14.4, 14.7 and 22.8±0.2 degrees.

7. Azilsartan medoxomil potassium crystalline Form II, characterized by an x-ray powder diffractogram as shown in FIG. 4.

8. A process for the preparation of azilsartan medoxomil potassium crystalline Form II as claimed in claim 6, which comprises:
a. suspending azilsartan medoxomil in a solvent;
b. heating the suspension obtained in step (a) at above 40° C.;
c. cooling the solution obtained in step (b) at room temperature;
d. adding potassium 2-ethylhexanoate in a solvent to the solution;
e. maintaining the reaction mass at room temperature; and
f. isolating azilsartan medoxomil potassium crystalline Form II.

9. The process as claimed in claim 8, wherein the solvent used in step (a) and step (d) is a solvent or mixture of solvents selected from acetone, methyl ethyl ketone, methyl isobutyl ketone, diethyl ketone, ethyl acetate, methyl acetate, isopropyl acetate, tert-butyl methyl acetate and ethyl formate.

10. The process as claimed in claim 9, wherein the solvents are acetone, methyl ethyl ketone and ethyl acetate.

11. The process as claimed in claim 8, wherein the reaction in step (b) is heated at about 45 to 65° C.

12. A pharmaceutical composition that comprises crystalline Form II of azilsartan acid and pharmaceutically acceptable excipients, and optionally other therapeutic ingredients.

13. A pharmaceutical composition that comprises crystalline Form II of azilsartan medoxomil potassium and pharmaceutically acceptable excipients, and optionally other therapeutic ingredients.

14. The pharmaceutical composition as claimed in claims 12 and 13, wherein the crystalline Forms are formulated into tablets, capsules, suspensions, dispersions or injectables.
